(19) United States
(12) Patent Application Publication
(10) Pub. No.: US 2024/0389867 A1
(43) Pub. Date: Nov. 28, 2024

(54) DETECTING RIGHT VENTRICULAR DYSFUNCTION IN CRITICAL CARE PATIENTS

(71) Applicant: EDWARDS LIFESCIENCES CORPORATION, Irvine, CA (US)

(72) Inventors: Cristhian M. Potes Blandon, Rancho Santa Margarita, CA (US); Kevin James Moses, Huntington Beach, CA (US); Christine Lee, Long Beach, CA (US)

(21) Appl. No.: 18/794,890

(22) Filed: Aug. 5, 2024

Related U.S. Application Data
(63) Continuation of application No. PCT/US2023/012363, filed on Feb. 5, 2023.
(60) Provisional application No. 63/307,252, filed on Feb. 7, 2022.

(51) Int. Cl. A61B 5/0205; A61B 5/00; A61B 5/01; A61B 5/02; A61B 5/021; A61B 5/0215; A61B 5/024; A61B 5/1455; G16H 50/30; G16H 50/70

(52) U.S. Cl. CPC A61B 5/02055; A61B 5/02028; A61B 5/02125; A61B 5/0215; A61B 5/14553; A61B 5/6814; A61B 5/6852; A61B 5/7239; A61B 5/7264; A61B 5/7275; A61B 5/7405; A61B 5/743; A61B 5/7455; A61B 5/746; G16H 50/30; G16H 50/70; A61B 5/01; A61B 5/024; A61B 2560/0462; A61B 2562/0271

(57) ABSTRACT

A system for monitoring hemodynamic data of a patient and providing a risk score representative of a likelihood of a right ventricular dysfunction event includes at least two hemodynamic sensors, a system memory, a user interface display, and a hardware processor. The hardware processor executes a right ventricular prediction software code stored within the system memory to receive hemodynamic data representative of a right ventricular pressure waveform of the patient and at least one of a pulmonary artery pressure waveform, a tissue oxygen saturation, a mixed venous oxygen saturation, and a cardiac output of the patient. Based on the hemodynamic data, the system determines and outputs the risk score to the display.

### CROSS-REFERENCE TO RELATED APPLICATION(S)

This application claims priority to the PCT Application having International Application No. PCT/US2023/012363, filed Feb. 5, 2023, and titled “DETECTING RIGHT VENTRICULAR DYSFUNCTION IN CRITICAL CARE PATIENTS,” which claims priority to U.S. Provisional Application No. 63/307,252, filed Feb. 7, 2022, and entitled “DETECTING RIGHT VENTRICULAR DYSFUNCTION IN CRITICAL CARE PATIENTS,” the disclosures of which are hereby incorporated by reference in their entireties.

### BACKGROUND

The present disclosure relates generally to hemodynamic monitoring of critically ill patients, and more specifically, to detecting current right ventricular dysfunction and predicting future right ventricular dysfunction.

Proper function of the right ventricle depends on the interplay between preload, contractility, afterload, ventricular interdependence, and heart rhythm. In some instances, right ventricular dysfunction occurs when increased isovolumic contraction time and isovolumic relaxation time lead to prolonged right ventricular systole and shortened right ventricular diastole. As right ventricular systole extends into left ventricular diastole, right ventricular volume loading reduces while right ventricular afterload increases, which leads to systolic right ventricular dysfunction. Diastolic dysfunction of the right ventricle occurs when contractile units do not return to their resting length.

Right ventricular dysfunction causes or worsens many illnesses and can be lethal in critically ill patients. One common cause of right ventricular dysfunction is acute pulmonary embolism, or a blockage of the pulmonary artery characterized by an excessive increase in afterload. Acute respiratory distress syndrome, in which fluid build-up within the lungs impairs oxygen delivery to the blood stream, is another condition that may be associated with right ventricular dysfunction. In other instances, right ventricular dysfunction can be a cause of death in patients experiencing pulmonary artery hypertension. Right ventricular dysfunction further complicates patients experiencing right ventricular myocardial infarction, which may be characterized by severe hypotension and low cardiac output. A variety of factors can contribute to right ventricular dysfunction in post-operative patients. These factors can include techniques undergone by the patient during surgy, such as cardiac bypass, and preexisting comorbidities, such as pulmonary vascular disease.

Depending on the cause of the right ventricular dysfunction, as well as current and preexisting conditions of the patient, hemodynamic indicia of right ventricular dysfunction may present differently. Because right ventricular dysfunction is often associated with or occurs contemporaneously with other patient illness, identification of prognostic indicators of right ventricular dysfunction can be difficult.

### SUMMARY

A system for monitoring hemodynamic data of a patient, in accordance with an exemplary embodiment of this disclosure, includes a first hemodynamic sensor, a second hemodynamic sensor, a system memory, a user interface, a display, and a hardware processor. The hardware processor executes a right ventricular prediction software code stored within the system memory to receive a first hemodynamic sensor signal representative of a right ventricular pressure waveform of the patient and a second hemodynamic sensor signal representative of a pulmonary artery pressure waveform, a tissue oxygen saturation, a mixed venous oxygen saturation, or a cardiac output of the patient. The hardware processor extracts at least one first waveform feature from the first hemodynamic sensor signal and determines the risk score based on the at least one first waveform feature and the second hemodynamic sensor signal. Thereafter, the hardware processor outputs the risk score to the display or the user interface.

A further embodiment of the system includes a hardware processor that determines the risk score based on the waveform feature extracted from the right ventricular pressure waveform and data representative of tissue oxygen saturation and the mixed venous oxygen saturation of the patient.

A further embodiment of the system includes a hardware processor that determines the risk score based on the waveform feature extracted from the right ventricular pressure waveform, the pulmonary artery pressure waveform, and data representative of cardiac output, tissue oxygen saturation, and the mixed venous oxygen saturation of the patient.

### BRIEF DESCRIPTION OF THE DRAWINGS

FIG. 1 is a perspective view of an example hemodynamic monitor that determines a risk score representing a probability of a future right ventricular dysfunction event for a patient.

FIG. 2 is a perspective view of a catheter that can be inserted in a patient and connected to one or more hemodynamic sensors.

FIG. 3 is a perspective view of an example minimally invasive pressure sensor for sensing hemodynamic data representative of pulmonary artery pressure or right ventricular pressure of a patient.

FIG. 4 is a perspective view of an oximetry module for receiving oximetry data from a catheter inserted within a patient.

FIG. 5A is a schematic view of a tissue oximetry sensor to determine oxygen saturation within cerebral tissue of a patient.

FIG. 5B is a tissue oximetry module that can be used in conjunction with a tissue oximetry sensor to determine oxygen saturation within cerebral tissue of a patient.

FIG. 6 is a block diagram illustrating an example hemodynamic monitoring system that determines a risk score representing a probability of a future right ventricular dysfunction event for a patient based on hemodynamic data.

FIG. 7 is a graph illustrating an example trace of a pulmonary artery pressure waveform including example indicia corresponding to the probability of a future right ventricular dysfunction event.

FIG. 8 is a graph illustrating an example trace of a right ventricular pressure waveform including example indicia corresponding to the probability of a future right ventricular dysfunction event.

FIG. 9 is a graph illustrating example traces of a pulmonary artery pressure waveform and a right ventricular pressure waveform including example indicia corresponding to the probability of a future right ventricular dysfunction event.

FIG. 10 is a diagram illustrating an example system for training a right ventricular dysfunction prediction model.

### DETAILED DESCRIPTION

As described herein, a hemodynamic monitoring system implements a predictive model that produces risk scores representing a probability of a current right ventricular dysfunction event for a patient, a probability of a future right ventricular dysfunction event for a patient, and a probability that the patient is experiencing a stable episode. The predictive model of the hemodynamic monitoring system uses machine learning to extract sets of input features from the right ventricular pressure and the pulmonary pressure of the patient in conjunction with data representative of tissue oxygen saturation and mixed venous oxygen saturation to produce the above-described risk scores for the patient during operation in, e.g., an operating room (OR), an intensive care unit (ICU), or other patient care environment. Depending on the level of the risk scores, the hemodynamic monitoring system can raise a signal or an alarm to medical workers to alert the medical workers that the patient is experiencing a right ventricular dysfunction event or soon will be experiencing a right ventricular dysfunction event. After receiving the signal, the medical workers can administer pharmaceuticals, or other medical care, to the patient to mitigate or prevent the right ventricular dysfunction event.

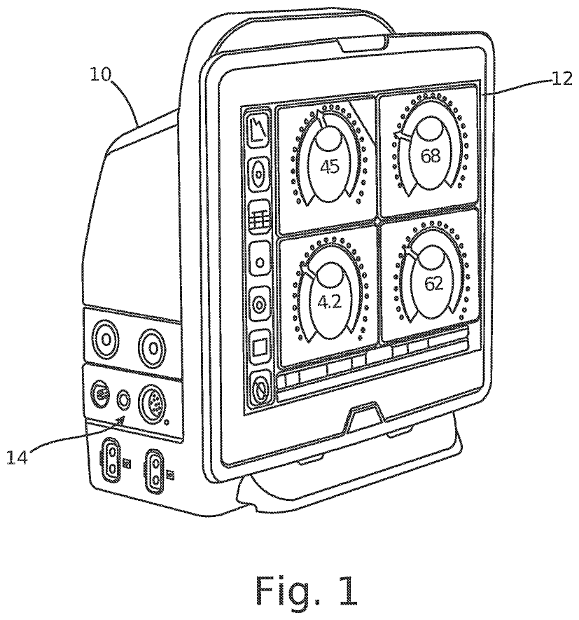

The machine learning of the predictive model of the hemodynamic monitoring system is trained using a clinical data set containing echocardiographic data, right ventricular pressure waveforms, pulmonary pressure waveforms, cardiac output data, tissue oxygen saturation data, and mixed venous oxygen saturation data. The hemodynamic monitoring system is described in detail below with reference to FIGS. 1-10.

FIG. 1 is a perspective view of hemodynamic monitor 10 that determines a risk score representing a probability of a current right ventricular dysfunction event of a patient and/or a risk score representing a probability of a future right ventricular dysfunction event for the patient. As illustrated in FIG. 1, hemodynamic monitor 10 includes display 12 that, in the example of FIG. 1, presents a graphical user interface including control elements (e.g., graphical control elements) that enable user interaction with hemodynamic monitor 10. Hemodynamic monitor 10 can also include a plurality of input and/or output (I/O) connectors configured for wired connection (e.g., electrical and/or communicative connection) with one or more peripheral components, such as one or more hemodynamic sensors, as is further described below. For instance, as illustrated in FIG. 1, hemodynamic monitor 10 can include I/O connectors 14. While the example of FIG. 1 illustrates five separate I/O connectors 14, it should be understood that in other examples, hemodynamic monitor 10 can include fewer than five I/O connectors or greater than five I/O connectors. In yet other examples, hemodynamic monitor 10 may not include I/O connectors 14, but rather may communicate wirelessly with various peripheral devices.

As further described below, hemodynamic monitor 10 includes one or more processors and computer-readable memory that stores right ventricular dysfunction detection and prediction software code which is executable to produce a risk score representing a probability of a present (i.e., current) right ventricular dysfunction event for a patient, a risk score representing a probability of a future right ventricular dysfunction event for the patient, and/or a risk score representing stable right ventricular function. Hemodynamic monitor 10 can receive sensed hemodynamic data representative of a right ventricular pressure waveform, a pulmonary pressure waveform, cardiac output, tissue oxygen saturation, and blood oxygen saturation of the patient, such as via one or more hemodynamic sensors connected to hemodynamic monitor 10 via I/O connectors 14. Hemodynamic monitor 10 executes the right ventricular dysfunction prediction software code to obtain, using the received hemodynamic data and multiple right ventricular dysfunction profiling parameters (e.g., input features), a risk score predictive of future right ventricular dysfunction as is further described below.

As illustrated in FIG. 1, hemodynamic monitor 10 can present a graphical user interface at display 12. Display 12 can be a liquid crystal display (LCD), a light-emitting diode (LED) display, an organic light-emitting diode (OLED) display, or other display device suitable for providing information to users in graphical form. In some examples, such as the example of FIG. 1, display 12 can be a touch-sensitive and/or presence-sensitive display device configured to receive user input in the form of gestures, such as touch gestures, scroll gestures, zoom gestures, swipe gestures, or other gesture input.

Hemodynamic monitor 10 receives hemodynamic data from a patient via one or more hemodynamic sensors 16A, 16B, 16C, and 16D (collectively hemodynamic sensors 16). In response to receiving hemodynamic data of the patient, hemodynamic monitor 10 executes the right ventricular dysfunction prediction software code to determine the risk score representing the probability of a current and/or future right ventricular dysfunction event for the patient and display the risk score on display 12. Additionally, hemodynamic monitor 10 can invoke a sensory alarm, such as an audible alarm, a haptic alarm, or other sensory alarm in response to determining that the risk score satisfies predetermined risk criteria. Accordingly, hemodynamic monitor 10 can provide a warning to medical personnel of a predicted future right ventricular dysfunction event of the patient prior to the patient entering right ventricular dysfunction or right ventricular failure.

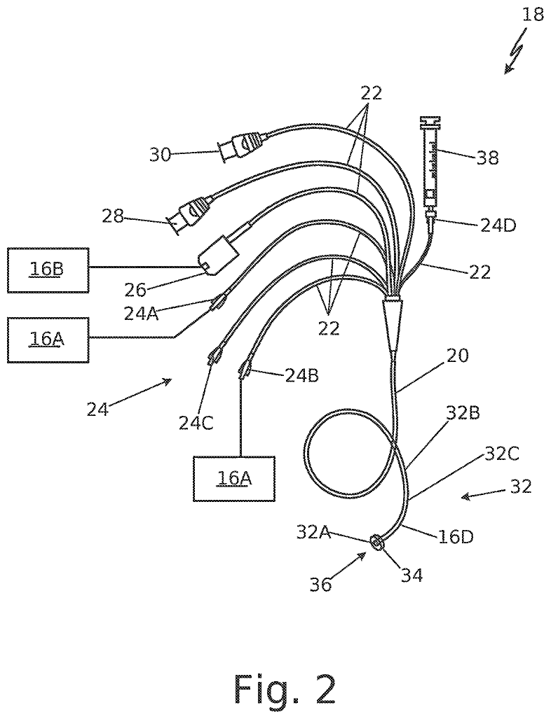

FIG. 2 depicts catheter 18 that can be connected to one or more hemodynamic sensors 16 for providing hemodynamic data to monitor 10. For example, catheter 18 may be connected to one or more pressure-sensing hemodynamic sensors 16A for detecting right ventricular pressure, pulmonary artery pressure, or both right ventricular and pulmonary artery pressures of the patient. Additionally, catheter 18 may interface with oximetry module 16B for sensing mixed venous oxygen saturation of the patient. Protected by sheath 20, catheter 18 includes multiple lumens 22 that place fluid connectors 24, optical connector 26, thermistor connector 28, and thermal filament connector 30 in communication with one of ports 32, an embedded hemodynamic sensor 16D (e.g., a thermistor), or thermal filament. To facilitate insertion of catheter 18 within patient, or for certain hemodynamic measurements, catheter 18 includes balloon 34 located at tip 36 of catheter 18.

As shown in FIG. 2, catheter 18 includes distal port connector 24A communicating with port 32A at tip 36. Proximal injectate connector 24B communicates with proximal port 32B disposed approximately 30 cm from tip 36 and can be used for dispensing fluids and drugs into the patient's heart. Right ventricular pacing connector 24C communicates with right ventricle port 32C spaced approximately 19 cm from tip 36. Connector 24C can be used for sensing a right ventricular pressure of the patient's heart. Thermistor connector 28 electrically connects to hemodynamic sensor 16D installed near tip 36 of catheter 18 for measuring core blood temperature within the pulmonary artery. In some embodiments of catheter 18, thermal filament connector 30 electrically connects to a filament embedded within catheter 18 located within the patient's right ventricle. Balloon connector 24D communicates with balloon 34 and with the use of syringe 38 can be used to inflate and deflate balloon 34.

After insertion into the patient, e.g., via an introducer, distal port connector 24A and right ventricular pacing connector 24C can be connected to separate pressure transducer sensors 16A. A first pressure transducer 16A provides pulmonary artery pressure waveform data to hemodynamic monitor 10 sensed at distal port 32A located within the pulmonary artery while a second pressure transducer 16A provides right ventricular pressure waveform data sensed at right ventricle port 32C located within the right ventricle of the patient's heart. Blood oxygen saturation data within the pulmonary artery can be provided by oximetry module 16B based on light pulses emitted from module 16B into the pulmonary artery and reflected light returns received by module 16B via optical connector 26 of catheter 18. Additionally, utilizing thermal filament connector 30 and thermistor connector 28 and associated cabling, hemodynamic monitor 10 can receive cardiac output data of the patient using, for example, a thermal dilution technique. If catheter 18 does not include a thermal filament, cardiac output can be determined using thermistor 28 after injecting a known volume and temperature of fluid via proximal injectate port 32B using the thermal dilution technique.

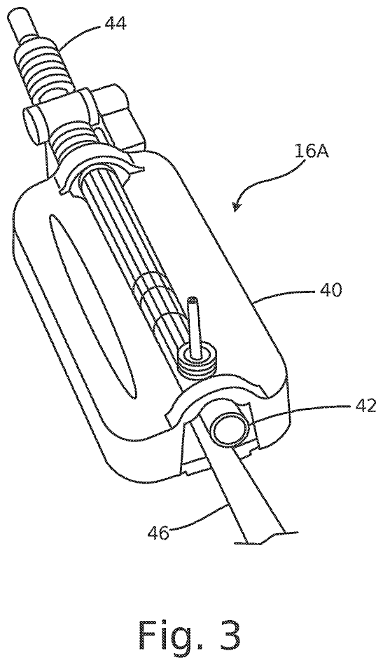

FIG. 3 is a perspective view of hemodynamic sensor 16A that can be attached to a patient for sensing hemodynamic data representative of right ventricular pressure or pulmonary artery pressure of the patient. As illustrated in FIG. 3, hemodynamic sensor 16A includes housing 40, fluid input port 42, catheter-side fluid port 44, and I/O cable 46. Fluid input port 42 is configured to be connected via tubing or other hydraulic connection to a fluid source, such as a saline bag or other fluid input source. Catheter-side fluid port 44 is configured to be connected via tubing or other hydraulic connection to a catheter (e.g., a radial arterial catheter or a femoral arterial catheter) that is inserted into an arm of the patient (i.e., a radial arterial catheter) or a leg of the patient (i.e., a femoral arterial catheter). I/O cable 46 is configured to connect to hemodynamic monitor 10 via, e.g., one or more of I/O connectors 14 (FIG. 1). Housing 40 of hemodynamic sensor 16A encloses one or more pressure transducers, communication circuitry, processing circuitry, and corresponding electronic components to sense fluid pressure corresponding to right ventricular pressure or pulmonary artery pressure of the patient that is transmitted to hemodynamic monitor 10 (FIG. 1) via I/O cable 46.

In operation, a column of fluid (e.g., saline solution) is introduced from a fluid source (e.g., a saline bag) through hemodynamic sensor 16A via fluid input port 42 to catheter-side fluid port 44 toward the catheter inserted into the patient. Right ventricular pressure or pulmonary artery pressure is communicated through the fluid column to pressure sensors located within housing 40 which sense the pressure of the fluid column. Hemodynamic sensor 16A translates the sensed pressure of the fluid column to an electrical signal via the pressure transducers and outputs the corresponding electrical signal to hemodynamic monitor 10 (FIG. 1) via I/O cable 46. Hemodynamic sensor 16 therefore transmits analog sensor data (or a digital representation of the analog sensor data) to hemodynamic monitor 10 (FIG. 1) that is representative of substantially continuous beat-to-beat monitoring of the right ventricular pressure or pulmonary artery pressure of the patient.

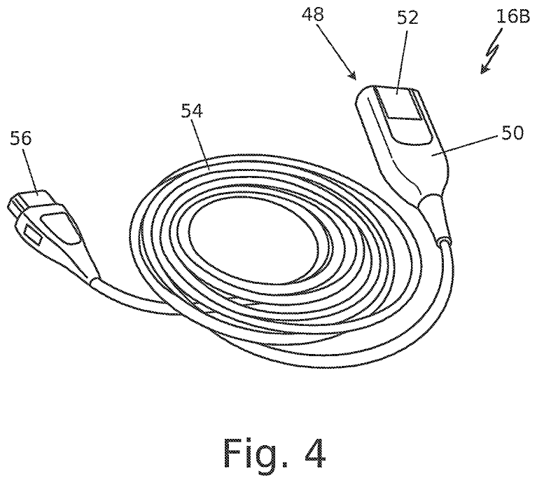

FIG. 4 depicts oximetry module 16B used for receiving oximetry data from a catheter inserted within a patient. As depicted in FIG. 4, hemodynamic sensor 16B includes an optical transmitter and an optical receiver arranged to communicate to a catheter via input/output connector 48 installed within housing 50 and accessible via protective door 52. Within housing 50, hemodynamic sensor 16B, as depicted by FIG. 4, includes communication circuitry, processing circuitry, and corresponding electronic components to sense blood oxygen saturation data derived from optical light emissions transmitted via a catheter into a patient and corresponding light returns received from the patient via the catheter. An electrical signal indicative of the patient blood oxygen saturation levels is transmitted to hemodynamic monitor 10 via cable 54 and connector 56, which interfaces with one of I/O connectors 14 (FIG. 1).

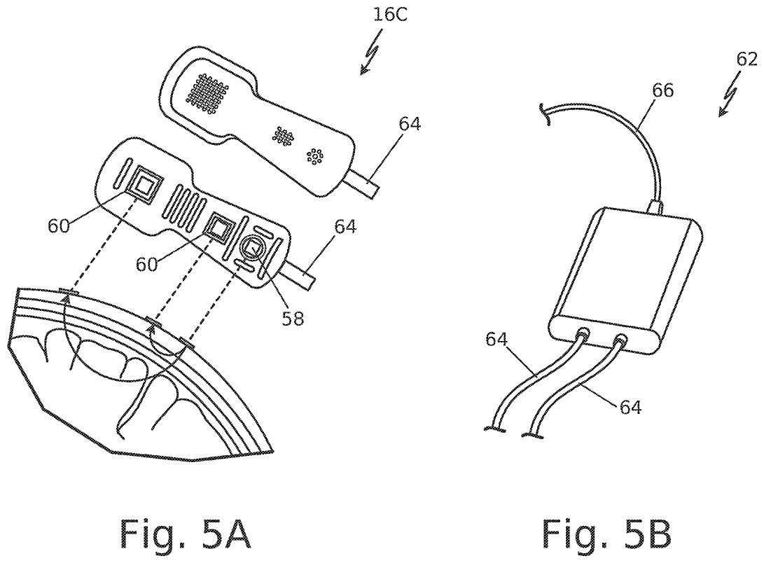

FIG. 5A is an isometric view of tissue oximetry sensor 16C for providing blood oxygen saturation data within cerebral tissue of the patient to hemodynamic monitor 10. Tissue oximetry sensor 16C includes light emitter 58 and one or more detectors 60. Oximetry module 62 depicted by the isometric view in FIG. 5B connects to one or more tissue oximetry sensors 16C via cable 64 and includes communication circuitry, processing circuitry, and corresponding electronic components to cause tissue oximetry sensor 16C or oximetry sensors 16C to emit light pulses into cerebral tissue of the patient. Light returns received by one or more detectors 60 of each tissue oximetry sensor 16C are received via cables 64 and processed by oximetry module 62. An electrical signal indicative of the patient tissue oxygen saturation levels is transmitted to hemodynamic monitor 10 via cable 66, which interfaces with one of I/O connectors 14 (FIG. 1).

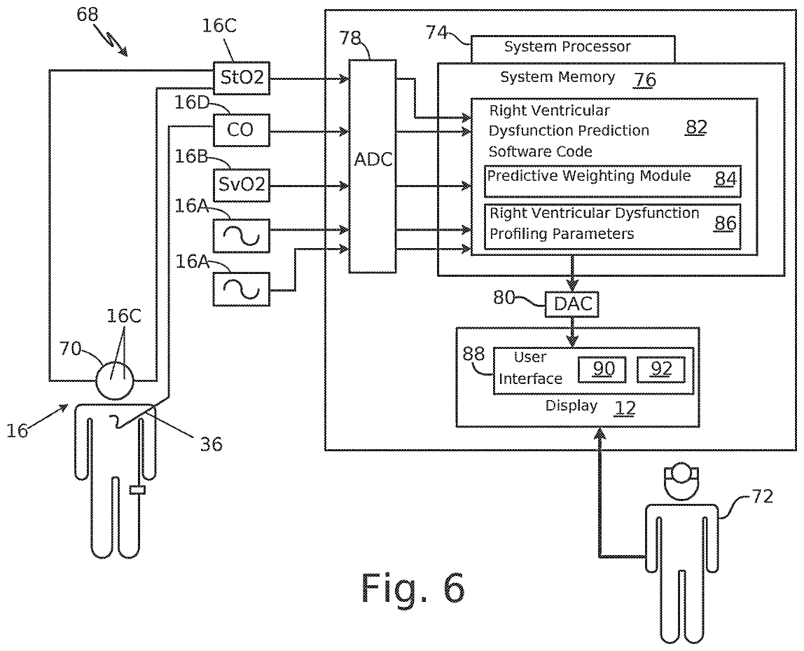

FIG. 6 is a block diagram of hemodynamic monitoring system 68 that determines a risk score representing a probability of a right ventricular dysfunction event based on hemodynamic data. As illustrated in FIG. 6, hemodynamic monitoring system 68 includes hemodynamic monitor 10 and hemodynamic sensors 16A, 16B, 16C, and 16D. Hemodynamic monitoring system 68 can be implemented within a patient care environment, such as an ICU, an OR, or other patient care environment. As illustrated in FIG. 6, the patient care environment can include patient 70 and healthcare worker 72 trained to utilize hemodynamic monitoring system 68.

Hemodynamic monitor 10, as described above with respect to FIG. 1, can be an integrated hardware unit including system processor 74, system memory 76, display 12, analog-to-digital (ADC) converter 78, and digital-to-analog (DAC) converter 80. In other examples, any one or more components and/or described functionality of hemodynamic monitor 10 can be distributed among multiple hardware units. For instance, in some examples, display 12 can be a separate display device that is remote from and operatively coupled with hemodynamic monitor 10. In general, though illustrated and described in the example of FIG. 6 as an integrated hardware unit, it should be understood that hemodynamic monitor 10 can include any combination of devices and components that are electrically, communicatively, or otherwise operatively connected to perform functionality attributed herein to hemodynamic monitor 10.

As illustrated in FIG. 6, system memory 76 stores right ventricular dysfunction prediction software code 82. Right ventricular dysfunction prediction software code 82 includes predictive weighting module 84 and right ventricular dysfunction profiling parameters 86. Display 12 provides user interface 88, which includes control elements 88 that enable user interaction with hemodynamic monitor 10 and/or other components of hemodynamic monitoring system 68. User interface 88, as illustrated in FIG. 6, also provides sensory alarm 92 to provide warning to medical personnel of a predicted future right ventricular dysfunction event of patient 70, as is further described below.

Hemodynamic sensors 16 can be attached to patient 70 to sense hemodynamic data representative of a right ventricular pressure waveform, a pulmonary artery pressure waveform, blood oxygen saturation, cerebral tissue oxygen saturation, or cardiac output of patient 70, or any combination of these hemodynamic data. Hemodynamic sensors 16 are operatively connected to hemodynamic monitor 10 (e.g., electrically and/or communicatively connected via wired or wireless connection, or both) to provide the sensed hemodynamic data to hemodynamic monitor 10. In some examples, hemodynamic sensors 16 provide the hemodynamic data of patient 70 to hemodynamic monitor 10 as an analog signal, which is converted by ADC 80 to digital hemodynamic data representative of the arterial pressure waveform. In other examples, hemodynamic sensors 16 can provide the sensed hemodynamic data to hemodynamic monitor 10 in digital form, in which case hemodynamic monitor 10 may not include or utilize ADC 78. In yet other examples, hemodynamic sensors 16 can provide the hemodynamic data of patient 70 to hemodynamic monitor 10 as an analog signal, which is analyzed in its analog form by hemodynamic monitor 10.

Hemodynamic sensors 16 can include a non-invasive, minimally invasive, or invasive sensor attached to patient 70. For instance, hemodynamic sensor 16 can take the form of invasive hemodynamic sensor 16A (FIG. 3), invasive hemodynamic sensor 16B (FIG. 4), non-invasive hemodynamic sensor 16C (FIGS. 5A and 5B) or other invasive, minimally invasive or non-invasive hemodynamic sensors. In some examples, hemodynamic sensors 16 can be attached non-invasively at an extremity of patient 70, such as a forehead, a wrist, an arm, a finger, an ankle, a toe, or other extremity of patient 70. In other examples, hemodynamic sensors 16 can be attached invasively to the patient, such as via catheter 18 (FIG. 4).

In certain examples, hemodynamic sensors 16 can be configured to sense right ventricular pressure, pulmonary artery pressure, or both right ventricular and pulmonary artery pressures of patient 70. In some instances, hemodynamic sensors 16 may also be used to sense cardiac output of the patient, blood oxygen saturation within the pulmonary artery, or both cardiac output and blood oxygen saturations in addition to right ventricular and pulmonary artery pressure waveforms. For instance, hemodynamic sensor 16 can be attached to patient 70 via a radial arterial catheter inserted into an arm of patient 70. In other examples, hemodynamic sensor 16 can be attached to patient 70 via a femoral arterial catheter inserted into a leg of patient 70. In other examples, hemodynamic sensor 16 may provide tissue oxygen saturation levels within cerebral tissue of patient 70 via an oximetry sensor attached to a forehead of patient 70. Such techniques can similarly enable multiple hemodynamic sensors 16 to provide substantially continuous beat-to-beat monitoring of the right ventricular pressure and pulmonary artery pressure as well as monitoring of cardiac output, blood oxygen saturation, and tissue oxygen saturation of patient 70, or any combination of these hemodynamic data, over an extended period of time, such as minutes or hours.

System processor 74 executes right ventricular dysfunction prediction software code 82, which implements predictive weighting module 84 utilizing right ventricular dysfunction profiling parameters 86 to produce a risk score representing a probability of a future right ventricular dysfunction event for patient 70. Examples of system processor 74 can include any one or more of a microprocessor, a controller, a digital signal processor (DSP), an application specific integrated circuit (ASIC), a field-programmable gate array (FPGA), or other equivalent discrete or integrated logic circuitry.

System memory 76 can be configured to store information within hemodynamic monitor 10 during operation. System memory 76, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 76 can include volatile and non-volatile computer-readable memories. Examples of volatile memories can include random access memories (RAM), dynamic random-access memories (DRAM), static random-access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

Display 12 can be a liquid crystal display (LCD), a light-emitting diode (LED) display, an organic light-emitting diode (OLED) display, or other display device suitable for providing information to users in graphical form. User interface 88 can include graphical and/or physical control elements that enable user input to interact with hemodynamic monitor 10 and/or other components of hemodynamic monitoring system 68. In some examples, user interface 88 can take the form of a graphical user interface (GUI) that presents graphical control elements presented at, e.g., a touch-sensitive and/or presence sensitive display screen of display 12. In such examples, user input can be received in the form of gesture input, such as touch gestures, scroll gestures, zoom gestures, or other gesture input. In certain examples, user interface 88 can take the form of and/or include physical control elements, such as a physical buttons, keys, knobs, or other physical control elements configured to receive user input to interact with components of hemodynamic monitoring system 68.

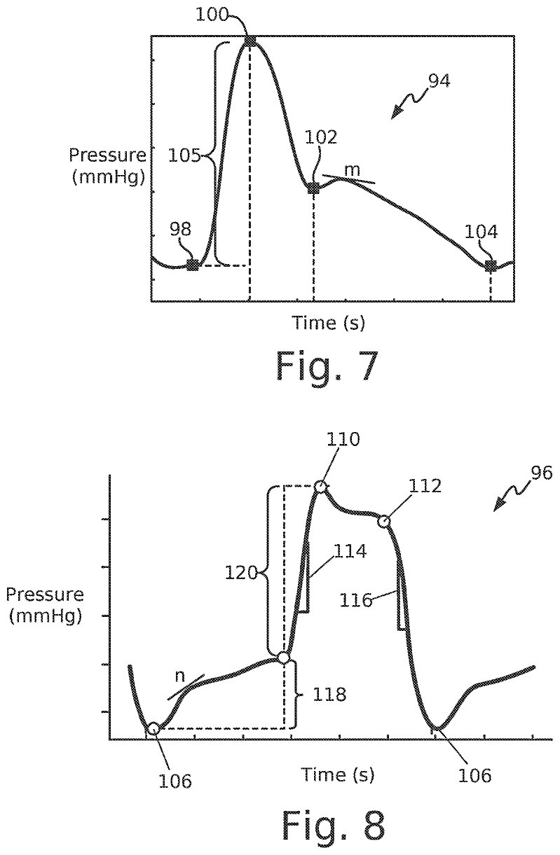

FIG. 7 is a graph illustrating an example trace of pulmonary artery pressure waveform 94 and, FIG. 8 is a graph illustrating an example trace of right ventricular pressure waveform 96, each corresponding to hemodynamic data sensed by one of hemodynamic sensors 16A and received by hemodynamic monitor 10. As further illustrated in FIGS. 7 and 8, pulmonary artery pressure waveform 94 and right ventricular pressure waveform 96 (e.g., represented via digital hemodynamic data) can include various indicia predictive of a future right ventricular dysfunction event for patient 70.

Prior to extracting indicia from pulmonary artery pressure waveform 94 and right ventricular, beat detector algorithms identify the start and end of individual heartbeats for each waveform. Pulmonary artery pressure beat detection algorithms identify the start of a heartbeat based on the maximum pulmonary artery pressure, the minimum pulmonary artery pressure, the maximum rate of change in pulmonary artery pressure, and/or the minimum rate of change in pulmonary artery pressure. Right ventricular pressure beat detection algorithms identify the start of the heartbeat based on the maximum right ventricular pressure, the minimum right ventricular pressure, the maximum or minimum rate of change in right ventricular pressure, and/or the second derivative with respect to time in the right ventricular pressure. After heartbeat identification within pulmonary artery pressure waveform 94 and right ventricular pressure waveform 96, various indicia of right ventricular dysfunction can be extracted from the waveforms on an on-going, beat-to-beat basis.

FIG. 7 illustrates example indicia 96, 100, 102, and 104, corresponding respectively to the start of a heartbeat (indicia 96), the maximum systolic pressure marking the end of systolic rise (indicia 100), the presence and pressure of the dicrotic notch marking the end of systolic decay (indicium 102), and the minimum diastolic pressure of the heartbeat (indicium 104) of patient 70. The mean pulmonary artery pressure can also be an indicium. Pulmonary artery pressure gradients, or pressure differences between points of the pulmonary artery pressure waveform 94, can be indicia. For instance, the difference between minimum diastolic pressure 104 and maximum systolic pressure 100 is known as the pulmonary pulse pressure (indicium 105.) Also shown in FIG. 7 is example slope “m” of pulmonary artery pressure waveform 94, though it should be understood that slope “m” is merely representative of multiple slopes that may be determined at multiple locations along pulmonary artery pressure waveform 94. For instance, example indicia may include the maximum and/or minimum time derivative of pulmonary artery pressure waveform 94.

Additional indicia predictive of right ventricular dysfunction for patient 70 can be extracted from pulmonary artery pressure waveform 94 by right ventricular dysfunction prediction software code 82 based on behavior of waveform 94 in various intervals, such as in the interval from the maximum systolic pressure at indicium 100 to the diastole at indicium 102, as well as the interval from the start of the heartbeat at indicium 96 to the diastole at indicium 104. Right ventricular dysfunction prediction software code 82 may identify indicia based on the behavior of pulmonary artery pressure waveform 94 during intervals: 1) systolic rise (indicium 96 to indicium 100), 2) systolic decay (indicium 100 to indicium 102), 3) systolic phase (indicium 96 to indicium 102), 4) diastolic phase (indicium 102 to indicium 104), 5) interval 100 to 104, and 6) heartbeat interval (between indicia 96) by determining the area under the curve of pulmonary artery pressure waveform 94 and the standard deviation of pulmonary artery pressure waveform 94 in each of intervals 1-6. The respective areas and standard deviations determined for intervals 1-6 can serve as additional indicia predictive of future right ventricular dysfunction event for patient 70. Additionally, the mean pulmonary artery pressure during one of intervals 1-6 may also be indicia.

FIG. 8 illustrates example indicia 106, 108, 110, and 112, corresponding respectively to the minimum diastolic pressure (indicium 106), end diastolic pressure (indicium 108), maximum systolic pressure (indicium 110), and end systolic pressure (indicium 112). The slope of right ventricular pressure waveform 96, as represented by slope “n”, may also provide indicia. As before, slope “n” is depicted at one location but is merely representative of multiple slopes that may be determined at multiple locations along right ventricular pressure waveform 96. For instance, the maximum pressure rate of change with respect to time (dP/dt) during systolic rise (indicium 114) and the minimum pressure rate of change with respect to time (dP/dt) during the relaxation period after end systole (indicium 116) are further example indicia. Similarly, the second time derivative of pressure waveform 96 may be determined at any location along right ventricular pressure waveform 96 and used as indicia. Other exemplary indicia include right ventricular pressure gradients, or the difference in pressure at different times during diastolic or systolic phase. For example, the diastolic gradient (indicium 118) is the difference between minimum diastolic pressure (indicium 106) and end diastolic pressure (indicium 108). Diastolic gradients 118 less than or equal to a threshold pressure, such as 4 mmHg or other threshold pressure, are indicative of normal right ventricular operation while diastolic gradients 118 greater than the threshold pressure (e.g., 4 mmHg) can be indicative of right ventricular dysfunction. Another right ventricular pressure gradient that can be indicative of right ventricular dysfunction is right ventricular pulse pressure 120. Right ventricular pulse pressure 120 of right ventricular pressure waveform 96 is a pressure gradient equal to the difference between end diastolic pressure (indicium 108) and maximum systolic pressure (indicium 110).

Additional indicia predictive of right ventricular dysfunction for patient 70 can be extracted from right ventricular pressure waveform 96 by right ventricular dysfunction prediction software code 82 based on behavior of right ventricular pressure waveform 96 during various intervals. For instance, the behavior of right ventricular pressure waveform 96 during intervals: 1) systolic rise (indicium 108 to indicium 110), 2) systolic decay (indicium 110 to indicium 111), 3) isovolumetric relaxation (indicium 112 to indicium 106), 4) diastolic phase (indicium 106 to indicium 109), and 5) heartbeat interval (between indicia 106), can be determined by right ventricular dysfunction prediction software code 82. Such indicia include the mean right ventricular pressure during one of intervals 1-5.

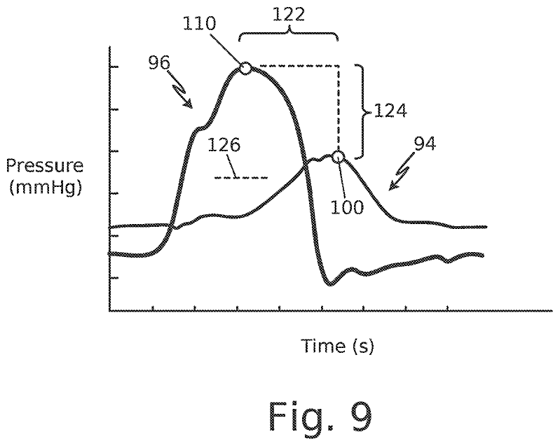

FIG. 9 illustrates example indicia that may be determined from differences between pulmonary artery pressure waveform 94 and right ventricular pressure waveform 96. For instance, the elapsed time between maximum systolic pressure (indicium 110) of right ventricular pressure waveform 96 and maximum systolic pressure (indicium 100) of pulmonary artery pressure waveform 94 is the pulse transit time (indicium 122). The pressure gradient between maximum systolic pressures (indicia 110 and 100) of pulmonary artery pressure waveform 94 and right ventricular pressure waveform 96 is the systolic gradient (indicium 124). The mean pressure of the pulmonary artery pressure waveform 94 and right ventricular pressure waveform 96 defines indicium 126. The pulmonary artery pulsatility index (PAPi) is pulmonary pulse pressure (indicium 105) divided by the mean right ventricular pressure and can be an indicator of right ventricular dysfunction. Another indicator of right ventricular dysfunction is right ventricular fraction index (RVFI), which is the right ventricular systolic pressure divided by the cardiac output.

Additional indicia of right ventricular dysfunction may include mixed venous oxygen saturation (SvO2), cerebral blood oxygen saturation (StO2), cardiac output, stroke volume, right ventricular end diastolic volume, right ventricular ejection fraction, pulse rate, and blood temperature, among other parameters determined or derived from pulmonary artery pressure waveform 94, right ventricular pressure waveform 96, cardiac output data, mixed venous oxygen saturation data, and cerebral tissue oxygen saturation data.

System processor 74 executes right ventricular dysfunction prediction software 82 to determine right ventricular dysfunction profiling parameters 86 based on indicia extracted or derived from the pulmonary artery pressure waveform, the right ventricular pressure waveform, tissue oxygen saturation data, blood oxygen saturation data, and cardiac output data. Predictive weighting module 84 applies risk coefficients, determined via training the predictive model, to profiling parameters 86. Based on the risk coefficients applied by predictive weighting module 84 and profiling parameters 86, right ventricular dysfunction prediction software code 82 determines the risk score representing a probability of a present or future right ventricular dysfunction event for patient 70.

The risk score can be a normalized value between 0 and 1 (or between 0 and 100, or other normalized ranges) with, in some examples, a higher value representing a higher likelihood that patient 70 will experience a right ventricular dysfunction event and a lower value representing a lower likelihood that patient 70 will experience a right ventricular dysfunction event. In another example, the normalized range of the risk scores can be subdivided into two or more continuous and sequential subranges, each subrange predictive of the patient's risk of experiencing a right ventricular dysfunction event. In some examples, the normalized range of risks scores can be subdivided into at least three continuous and sequential subranges. The first of the three subranges (e.g., a risk score value between 0 and 30 within a normalized range from 0 to 100) can be indicative of a stable patient. The second subrange (e.g., a risk score between 31 and 60 within a normalized range from 0 to 100) can be predictive of potential future right ventricular dysfunction. The third subrange (e.g., a risk score value between 61 and 100 within a normalized range from 0 to 100) can be indicative of current right ventricular dysfunction and/or predictive of future right ventricular failure of the patient.

Sensory alarm 92 can be configured to be invoked if, for example, the risk score is greater than 0.60 (when measured on a normalized scale of 0 to 1) or 60 (when measured on a normalized scale of 0 to 100). Sensory alarm 92 can be implemented as one or more of a visual alarm, an audible alarm, a haptic alarm, or other type of sensory alarm. For instance, sensory alarm 92 can be invoked as any combination of flashing and/or colored graphics shown by user interface 88 on display 12, display of the risk score via user interface 88 on display 12, a warning sound such as a siren or repeated tone, and a haptic alarm configured to cause hemodynamic monitor 10 to vibrate or otherwise deliver a physical impulse perceptible to healthcare worker 72 or other user.

Accordingly, hemodynamic monitor 10 provides a warning to medical personnel of a likelihood that patient 70 will experience a right ventricular dysfunction event with the determination of the risk score and the potential warning occurring before the onset of right ventricular dysfunction, thereby enabling timely and effective intervention to prevent (or mitigate) the right ventricular dysfunction event that may occur. Techniques described herein therefore increase the usability of hemodynamic monitor 10 by enabling hemodynamic monitor 10 to determine, before the onset of right ventricular dysfunction, the likelihood that patient 70 will experience a right ventricular dysfunction event.

One example of an equation for determining the right ventricular dysfunction risk score is as follows:

$$Risk Score = \frac{100}{1 + {e}^{- ( {w}_{0} + \sum {w}_{i} {x}_{i} )}}$$

$$for$$

$$i = 1 , 2 , 3 , … , 13$$

model)
   where, xᵢ's are the example indicia determined from pulmonary artery pressure waveform 94, right ventricular pressure waveform 96, cardiac output data, tissue oxygen saturation data, and mixed venous oxygen saturation data (examples listed below), wᵢ's are the corresponding feature weights (i.e., coefficients), and wo is a bias term.
x₁=The mean right ventricular pressure
x₂=The maximum right ventricular pressure
x₃=The minimum right ventricular pressure
x₄=The right ventricular pulse pressure
x₅=The maximum rate of right ventricular pressure change with respect to time during systolic rise
x₆=The minimum rate or right ventricular pressure change with respect to time during the relaxation period after end systole
x₇=The right ventricular end diastolic pressure
x₈=The right ventricular diastolic gradient
x₉=The systolic pressure gradient
x₁₀=The pulse transit time
x₁₁=The right ventricular function index
x₁₂=The mixed venous oxygen saturation
x₁₃=The cerebral blood oxygen saturation
x₁₄=The cardiac output
x₁₅=The stroke volume
x₁₆=The right ventricular end diastolic volume
x₁₇=The right ventricular ejection fraction
x₁₈=The pulse rate
x₁₉=The blood temperature
x₂₀=The mean pulmonary artery pressure
x₂₁=The arterial elastance
x₂₂=The maximal elastance
x₂₃=The ventriculoarterial coupling
x₂₄=The pulmonary vascular resistance
   The right ventricular dysfunction risk score can be determined using one, a selected number, or all of the indicia, and some examples of determining the right ventricular dysfunction risk score can include the use of other indicia from pulmonary artery pressure waveform 94, right ventricular pressure waveform 96, cardiac output data, tissue oxygen saturation data, and blood oxygen saturation data features other than those set out above.

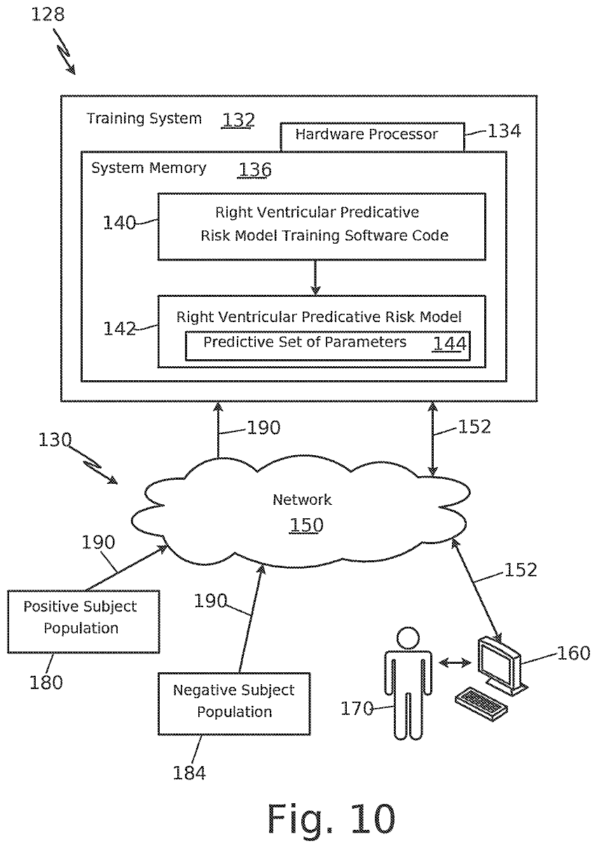

FIG. 10 shows a diagram illustrating example training system 128 for training a right ventricular dysfunction predictive risk model. Training system 128 is situated within communication environment 130 including communication network 150, client system 160, system user 170, population of positive subjects 180, and population of negative subjects 184. Training system 132 includes hardware processor 134 and system memory 136 for storing right ventricular dysfunction predictive risk model training software code 140. System memory 136 can include right ventricular dysfunction predictive risk model 142 including predictive set of parameters 144, which can be right ventricular pressure waveform indicia, pulmonary artery pressure waveform indicia, cardiac output data indicia, tissue oxygen saturation indicia, mixed venous oxygen saturation data indicia arterial, and patient demographic features/information. Network communication links 152 interactively connect client system 160 to training system 132 via communication network 150 and allow for the transmission of hemodynamic data 190 (which can include echocardiographic data, pulmonary artery pressure waveform, right ventricular pressure waveform, cardiac output data, tissue oxygen saturation data, and mixed venous oxygen saturation data) from population of positive subjects 180 and population of negative subjects 184 to training system 132 via communication network 150.

System user 170 (who may be a medical professional, health care worker, or medical researcher) may utilize client system 160 to interact with training system 132 over communication network 150. For example, system user 170 may receive right ventricular dysfunction predictive risk model 142 (including predictive set of parameters 144) over communication network 150 and/or may download right ventricular dysfunction predictive risk model training software code 140 to client system 160 via communication network 150. In one implementation, training system 132 may correspond to one or more web servers with accessibility over a packet network, such as the internet. Alternatively, training system 132 may correspond to one or more servers supporting a local area network (LAN) or included in another type of limited distribution network.

Hardware processor 134 is configured to execute right ventricular dysfunction risk model training software code 140 to receive hemodynamic data 190 of each subject of population of positive subjects 180 and each subject of population of negative subjects 184 with hemodynamic data 190 being collected for a period of time. In positive subjects 180, the patient experiences a right ventricular dysfunction event, and in negative subjects 184, the patient does not experience a right ventricular dysfunction event.

Criteria that define right ventricular dysfunction, and thereby distinguish positive subjects 180 from negative subjects 184, are established by a panel of clinicians. While criteria can be derived from any subset of hemodynamic data 190 representative of positive subjects 180 and/or negative subjects 184, echocardiographic data of positive subjects 180 and negative subjects 194 are particularly useful. Echocardiographic techniques for identifying right ventricular dysfunction include visual examination of the right ventricle via 2D and/or 3D echocardiographic imaging, tricuspid annular plane systolic excursion (TAPSE), tissue doppler of the free lateral wall (S′), and fractional area change (FAC).

Echocardiographic indicators obtained by visual examination of the right ventricle include transverse dimension at the base and mid-level as well as the longitudinal dimension of the right ventricle. Additional visual indications include the proximal diameter of the right ventricular outflow tract measured in the parasternal short axis view and/or in the parasternal long axis view and the distal diameter of the right ventricle outflow tract measured at the level of pulmonary valve insertion.

Tricuspid annular plane systolic excursion (TAPSE) measures the maximum systolic excursion of the lateral tricuspid annulus. Lower TAPSE valves (i.e., less than 17 mm) are indicative of right ventricular dysfunction. Tissue doppler of the free lateral wall (S′) measures the longitudinal velocity (base to apex) of the tricuspid annular plane by tissue Doppler imaging. Again, lower S′ values (e.g., less than 0.095 m/s) are indicative of right ventricular dysfunction.

Fractional area change (FAC) is a two-dimensional representation of right ventricular ejection fraction (RVEF). To obtain the fraction area change (FAC), the right ventricle borders are traced during systole and diastole. From this data, the fractional area change of the right ventricle can be determined according to the equation below.

$$FAC = ( \frac{Area of RV at end diastic - Area of RV at end systolic}{Area of RV at end diastic} ) 100$$

Based on one or more of the preceding criteria, or another subset of hemodynamic data 190, clinicians identify positive subjects 180 and negative subjects 184. These clinicians associate the remaining hemodynamic data 190 (e.g., right ventricular waveform data, pulmonary artery waveform data, cardiac output data, tissue oxygen saturation data, and/or blood oxygen saturation data) with positive subjects 180 and/or negative subjects 184.

Subsequently, hardware processor 134 is further configured to execute right ventricular dysfunction risk model training software code 140 to define hemodynamic data 190 sets for use in training the right ventricular dysfunction risk model and extract waveform features from the pulmonary artery pressure waveform, the right ventricular pressure waveform, cardiac output data, tissue oxygen saturation data, and mixed venous oxygen saturation data (of the hemodynamic data 190 sets) of the positive subject 190. In addition, hardware processor 134 is configured to execute right ventricular dysfunction risk model training software code 140 to transform the waveform features, cardiac output data, and oximetry data from positive subjects 190 to a plurality of parameters. Right ventricular dysfunction risk model training software code 140 then identifies, from the plurality of parameters, predictive set of parameters 144 enabling prediction that the patient will experience the right ventricular dysfunction event after (e.g., identifies the hemodynamic data indicia that are most indicative in predicting a right ventricular dysfunction event). The plurality of parameters characterizing the waveform features (extracted from the hemodynamic data) can be one, a combination of, or all of the mean right ventricular pressure, the maximum right ventricular pressure, the minimum right ventricular pressure, the right ventricular pulse pressure, the maximum rate of right ventricular pressure change with respect to time during systolic rise, the minimum rate or right ventricular pressure change with respect to time during the relaxation period after end systole, the right ventricular end diastolic pressure, the right ventricular diastolic gradient, the systolic pressure gradient, the pulse transit time, the right ventricular function index, the mixed venous oxygen saturation, the cerebral blood oxygen saturation, the cardiac output, the stroke volume, the right ventricular end diastolic volume, the right ventricular ejection fraction, the pulse rate, the blood temperature, the mean pulmonary artery pressure, the arterial elastance, the maximal elastance, the ventriculoarterial coupling, and the pulmonary vascular resistance. Additionally, the plurality of parameters can also include patient demographic information/features, such as an age, gender, height, weight, and physical status classification score of the positive subject.

Identifying predictive set of parameters 144 from the plurality of parameters can include obtaining differential parameters based on the plurality of parameters characterizing the right ventricular pressure waveform features, the pulmonary artery pressure waveform features, blood oxygen saturation data, tissue oxygen saturation data, and/or cardiac output data. Further, identification of predictive set of parameters 144 can include generating combinatorial parameters and/or generating inter-relationship parameters over short periods of time using the plurality of parameters characterizing the waveform features, oximetry data, or cardiac output data as well as any associated differential parameters. The differential parameters can be the same, partially the same, or different parameters than the plurality of parameters. Predictive set of parameters 144 can then be identified from the plurality of parameters, the differential parameters, the inter-relational parameters, and the combinatorial parameters to select a reduced set of parameters that are most indicative of predicting a right ventricular dysfunction event. The combinatorial parameters can be a power combination of all or a subset of the plurality of parameters and the differential parameters, and the power combinations can include integer powers from among, for example, negative two, negative one, positive one, and/or positive two.

Hardware processor 134 can also be configured to execute right ventricular dysfunction risk model training software code 140 to identify, from among the reduced set of parameters, predictive set of parameters 144 more correlated to the occurrence of a right ventricular dysfunction event, thereby training right ventricular predictive risk model 142. From the predictive set of parameters 144, hardware processor 134 can be configured to execute right ventricular predictive risk model training software code 140 to compute predictive risk model coefficients corresponding to the predictive set of parameters to minimize the error of the right ventricular dysfunction score outputted by right ventricular predictive risk model 142, thereby further training right ventricular predictive risk model 142 to minimize error.

Right ventricular dysfunction predictive risk model 142 (and right ventricular dysfunction risk model training software code 140), can be a machine learning model that is an artificial neural network model, a machine learning model that is a known nearest neighbor model, a machine learning model that utilizes linear regression to identify predictive set of parameters 144 and determine the predictive risk model coefficients, or another type of model for identifying predictive set of parameters 144 and determining the predictive risk model coefficients that most accurately represent the likelihood that a patient will experience a right ventricular dysfunction event.

In some implementations, hardware processor 134 is configured to execute right ventricular dysfunction predictive risk model training software code 140 to display right ventricular dysfunction predictive risk model 142, the plurality of parameters characterizing hemodynamic data 190, and or predictive set of parameters 144 to system user 170 through display features available on client system 160. Additionally, hardware processor 134 is configured to execute right ventricular dysfunction predictive risk model training software code 140 to update or otherwise modify predictive set of parameters 144 and/or predictive risk model coefficients based on additional hemodynamic data 190 and/or patient demographic information/features received from one or more positive subjects of the population of positive subjects 180 and negative subjects of the population of negative subjects 184.

For example, training system 132 can receive additional hemodynamic data from one or more negative subjects from the population of negative subjects 184 (subjects that did not experience a right ventricular dysfunction event). Hardware processor 134 can then execute right ventricular dysfunction predictive risk model training software code 140 to extract predictive set of parameters 144 (e.g., waveform features and/or patient demographic information/features) from the hemodynamic data with predictive set of parameters 144 being similar to predictive set of parameters 144 identified with respect to positive subject 180. Hardware processor 134 can then execute right ventricular dysfunction predictive risk model training software code 140 to determine the right ventricular dysfunction score utilizing the same predictive risk model coefficients previously calculated and compare that right ventricular dysfunction score to a baseline right ventricular dysfunction score for a negative subject that did not experience a right ventricular dysfunction event. If the right ventricular dysfunction score is not within a margin of error of the baseline right ventricular dysfunction score, hardware processor 134 can then execute right ventricular dysfunction predictive risk model training software code 140 to alter predictive set of parameters 144 and the predictive risk model coefficients to more accurately predict the likelihood that a right ventricular dysfunction event will occur, and training system 132 can then repeat the training steps with additional hemodynamic data from positive subjects 180 and/or negative subjects 184.

Although FIG. 10 shows right ventricular dysfunctions risk model 142 as residing in or otherwise a part of system memory 136, other configurations can include a right ventricular dysfunction predictive risk model that is copied to non-volatile storage (not shown in FIG. 10) or may be transmitted to client system 160 via communication network 150. Further, though client system 160 is shown as a personal computer in FIG. 10, such a configuration is provided merely as an example and other configurations may include client system 160 as or within a mobile communication device, such as a smartphone or tablet computer.

As described above, a system for determining a right ventricular dysfunction score, such as a hemodynamic monitoring system, and related methods of determining the right ventricular dysfunction score and training a right ventricular dysfunction predictive risk model to determine the right ventricular dysfunction score produce a right ventricular dysfunction score that represents the likelihood that a patient will experience a right ventricular dysfunction event. The right ventricular dysfunction score is determined based on hemodynamic data, such as pulmonary artery pressure waveform data, the right ventricular pressure waveform data, the cardiac output data, the tissue oxygen saturation data, and mixed venous oxygen saturation data collected by one or more hemodynamic sensors 16, during a period of time before the patient experiences a right ventricular dysfunction event. The right ventricular dysfunction score is determined and conveyed to a medical professional so that the medical professional has a warning that the patient is likely (or not likely) to experience a right ventricular dysfunction event.

The right ventricular dysfunction score is determined based on a weighted combination of right ventricular dysfunction parameters, for example a predictive set of parameters that include waveform features extracted from the hemodynamic data and/or patient demographic information, that are predictive of the future right ventricular dysfunction event. The selection of risk coefficients and/or the predictive set of parameters can be accomplished via training (e.g., offline training) of the right ventricular dysfunction predictive risk model using machine learning or other techniques to minimize the error of the predictive risk model output (i.e., the right ventricular dysfunction score).

### Discussion of Possible Embodiments

The following are non-exclusive descriptions of possible embodiments of the present invention.

A system for monitoring hemodynamic data of a patient and providing a risk score representative of a likelihood of a right ventricular dysfunction event, the system comprising: a first hemodynamic sensor that produces, on an ongoing basis, a first hemodynamic sensor signal representative of a right ventricular pressure waveform of the patient; a second hemodynamic sensor that produces, on an ongoing basis, a second hemodynamic sensor signal representative of one of a pulmonary artery waveform, a tissue oxygen saturation, a mixed venous oxygen saturation, and a cardiac output of the patient; a system memory that stores a right ventricular prediction software code; a user interface that includes a display; and a hardware processor that is configured to execute the right ventricular prediction software code to: receive the first hemodynamic sensor signal representative of the right ventricular pressure waveform of the patient; receive the second hemodynamic sensor signal representative of the pulmonary artery waveform, the tissue oxygen saturation, the mixed venous oxygen saturation, or the cardiac output of the patient; extract at least one first waveform feature from the right ventricular pressure waveform of the patient; determine the risk score based on the at least one first waveform feature and the second hemodynamic sensor signal; and output the risk score to the display,

The system of the preceding paragraph can optionally include, additionally and/or alternatively, any one or more of the following features, configurations and/or additional components:

Extracting the at least one first waveform feature includes determining at least one of or any combination of a mean right ventricular pressure, a maximum right ventricular systolic pressure, a minimum right ventricular diastolic pressure, a right ventricular end diastolic pressure, a right ventricular end systolic pressure, a maximum first derivative with respect to time of the right ventricular pressure waveform, and a minimum first derivative with respect to time of the right ventricular pressure waveform.

The hardware processor executes the right ventricular prediction software to: determine a right ventricular pulse pressure equal to a difference between the maximum right ventricular systolic pressure and the right ventricular end diastolic pressure; and determine the risk score based on the at least one first waveform feature and the right ventricular pulse pressure.

The hardware processor executes the right ventricular prediction software to: determine a right ventricular pulse pressure variation as a beat-to-beat difference in the right ventricular pulse pressure; and determine the risk score based on the at least one first waveform feature, the right ventricular pulse pressure, and the right ventricular pulse pressure variation.

The hardware processor executes the right ventricular prediction software to: determine a right ventricular diastolic gradient equal to a difference between a right ventricular end diastolic pressure and a minimum right ventricular diastolic pressure; and determine the risk score based on the at least one first waveform feature and the right ventricular diastolic gradient.

The second hemodynamic sensor signal is representative of the pulmonary artery waveform of the patient, and wherein the hardware processor executes the right ventricular prediction software to: extract at least one second waveform feature from the pulmonary artery waveform of the patient; and determine the risk score based on the at least one first waveform feature and the at least one second waveform feature.

Extracting the at least one second waveform feature includes determining at least one of or any combination of a maximum pulmonary artery systolic pressure, a minimum pulmonary artery diastolic pressure, a mean pulmonary artery pressure, a pulmonary artery pressure at the end of systolic decay, a maximum first derivative with respect to time of the pulmonary artery pressure waveform, and a minimum first derivative with respect to time of the pulmonary artery pressure waveform.

The second waveform feature is a pulmonary artery pulse pressure equal to a difference between the maximum pulmonary artery systolic pressure and the minimum pulmonary artery diastolic pressure, and wherein the hardware processor executes the right ventricular prediction software to determine the risk score based on the at least one first waveform feature and the pulmonary artery pulse pressure.

The second waveform feature is a maximum pulmonary artery systolic pressure, and wherein the hardware processor executes the right ventricular prediction software to: determine a pulse transit time equal to an elapsed time between a maximum right ventricular systolic pressure and the maximum pulmonary artery systolic pressure and determine the risk score based on the at least one first waveform feature and the pulse transit time.

The second waveform feature is a maximum pulmonary artery systolic pressure, and wherein the hardware processor executes the right ventricular prediction software to: determine a systolic gradient equal to a pressure difference between a maximum right ventricular systolic pressure and the maximum pulmonary artery systolic pressure; and determine the risk score based on the at least one waveform feature and the systolic gradient.

A third hemodynamic sensor that produces, on an ongoing basis, a third hemodynamic sensor signal representative of one of a mixed venous oxygen saturation, a tissue oxygen saturation, and a cardiac output of the patient, wherein the hardware processor executes the right ventricular prediction software code to determine the risk score based on the at least one first waveform feature, the at least one second waveform feature, and the third hemodynamic sensor signal.

A fourth hemodynamic sensor that produces, on an ongoing basis, a fourth hemodynamic sensor signal representative of a tissue oxygen saturation of the patient, wherein the third hemodynamic sensor signal is representative of the mixed venous oxygen saturation of the patient, and wherein the hardware processor executes the right ventricular prediction software code to determine the risk score based on the at least one first waveform feature, the at least one second waveform feature, the third hemodynamic sensor signal, and the fourth hemodynamic sensor signal.

A fifth hemodynamic sensor that produces, on an ongoing, bases a fifth hemodynamic sensor signal representative of a cardiac output of the patient, wherein the hardware processor executes the right ventricular prediction software code to determine the risk score based on the at least one first waveform feature, the at least one second waveform feature, the third hemodynamic sensor signal, the fourth hemodynamic sensor signal, and the fifth hemodynamic sensor signal.

A catheter inserted within a pulmonary artery and the right ventricle of the patient, wherein the first hemodynamic sensor, the second hemodynamic sensor, and the third hemodynamic senor are connected to the catheter, and wherein the fifth hemodynamic sensor includes a thermistor embedded within the catheter.

The fourth hemodynamic sensor is a brain tissue oximetry sensor, and wherein the fourth sensor signal is representative of a cerebral tissue oxygen saturation of the patient.

The hardware processor executes the right ventricular prediction software code to: determine at least one of or any combination of a continuous cardiac output, a stroke volume, a right ventricular ejection fraction, a right ventricular end diastolic volume, a pulse rate, and a blood temperature; and determine the risk score based on the at least one first waveform feature, the at least one second waveform feature, the third hemodynamic sensor signal, the fourth hemodynamic sensor signal, the fifth hemodynamic sensor signal, and the at least one of or any combination of a continuous cardiac output, a stroke volume, a right ventricular ejection fraction, a right ventricular end diastolic volume, a pulse rate, and a blood temperature.

The hardware processor executes the right ventricular prediction software code to: determine at least one of or any combination of an arterial elastance, a maximal elastance, a ventriculoarterial coupling, a pulmonary vascular resistance, a pulmonary artery pulsatility index, a right ventricular function index; and determine the risk score based on the at least one waveform feature, the at least one second waveform feature, the third hemodynamic sensor signal, the fourth hemodynamic sensor signal, the fifth hemodynamic sensor signal, and the at least one of or any combination of the arterial elastance, the maximal elastance, the ventriculoarterial coupling, the pulmonary vascular resistance, the pulmonary artery pulsatility index, the right ventricular function index.

The hardware processor executes the right ventricular prediction software code to determine the risk score based on: at least one of or any combination of a right ventricular end diastolic pressure, a right ventricular diastolic gradient, and a minimum first derivative with respect to time of the right ventricular pressure waveform; at least one of a maximum first derivative with respect to time of the right ventricular pressure waveform, a right ventricular systolic pressure, a right ventricular end systolic pressure, and a right ventricular pulse pressure; at least one of or any combination of a right ventricular diastolic pressure and a right ventricular pulse pressure variation; at least one of or any combination of a pulse transit time, a mean pulmonary artery pressure, and a systolic gradient; and at least one of or any combination of a mixed venous oxygen saturation and a cerebral oxygen saturation.

The hardware processor executes the right ventricular prediction software code to subdivide a range of risk scores into at least three continuous and sequential subranges, wherein a first subrange is predictive of right ventricular dysfunction of the patient, and wherein a second subrange of risk scores is indicative of potential right ventricular dysfunction, and wherein a third subrange of risk scores is indicative of a stable patient.

The user interface includes a sensory alarm, and wherein the hardware processor executes the right ventricular prediction software code to activate the sensory alarm when the risk score is within the first subrange.

While the invention has been described with reference to an exemplary embodiment(s), it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment(s) disclosed, but that the invention will include all embodiments falling within the scope of the appended claims.

1. A system for monitoring hemodynamic data of a patient and providing a risk score representative of a likelihood of a right ventricular dysfunction event, the system comprising:
a first hemodynamic sensor that produces, on an ongoing basis, a first hemodynamic sensor signal representative of a right ventricular pressure waveform of the patient;
a second hemodynamic sensor that produces, on an ongoing basis, a second hemodynamic sensor signal representative of one of a pulmonary artery waveform, a tissue oxygen saturation, a mixed venous oxygen saturation, and a cardiac output of the patient;
a system memory that stores a right ventricular prediction software code;
a user interface that includes a display; and
a hardware processor that is configured to execute the right ventricular prediction software code to:
receive the first hemodynamic sensor signal representative of the right ventricular pressure waveform of the patient;
receive the second hemodynamic sensor signal representative of the pulmonary artery waveform, the tissue oxygen saturation, the mixed venous oxygen saturation, or the cardiac output of the patient;
extract at least one first waveform feature from the right ventricular pressure waveform of the patient;
determine the risk score based on the at least one first waveform feature and the second hemodynamic sensor signal; and
output the risk score to the display.

2. The system of claim 1, wherein extracting the at least one first waveform feature includes determining at least one of a mean right ventricular pressure, a maximum right ventricular systolic pressure, a minimum right ventricular diastolic pressure, a right ventricular end diastolic pressure, a right ventricular end systolic pressure, a maximum first derivative with respect to time of the right ventricular pressure waveform, and a minimum first derivative with respect to time of the right ventricular pressure waveform.

3. The system of claim 2, wherein the hardware processor executes the right ventricular prediction software to:
determine a right ventricular pulse pressure equal to a difference between the maximum right ventricular systolic pressure and the right ventricular end diastolic pressure; and
determine the risk score based on the at least one first waveform feature and the right ventricular pulse pressure.

4. The system of claim 3, wherein the hardware processor executes the right ventricular prediction software to:
determine a right ventricular pulse pressure variation as a beat-to-beat difference in the right ventricular pulse pressure; and
determine the risk score based on the at least one first waveform feature, the right ventricular pulse pressure, and the right ventricular pulse pressure variation.

5. The system of claim 1, wherein the hardware processor executes the right ventricular prediction software to:
determine a right ventricular diastolic gradient equal to a difference between a right ventricular end diastolic pressure and a minimum right ventricular diastolic pressure; and
determine the risk score based on the at least one first waveform feature and the right ventricular diastolic gradient.

6. The system of claim 1, wherein the second hemodynamic sensor signal is representative of the pulmonary artery waveform of the patient, and wherein the hardware processor executes the right ventricular prediction software to:
extract at least one second waveform feature from the pulmonary artery waveform of the patient; and
determine the risk score based on the at least one first waveform feature and the at least one second waveform feature.

7. The system of claim 6, wherein extracting the at least one second waveform feature includes determining at least one of a maximum pulmonary artery systolic pressure, a minimum pulmonary artery diastolic pressure, a mean pulmonary artery pressure, a pulmonary artery pressure at the end of systolic decay, a maximum first derivative with respect to time of the pulmonary artery pressure waveform, and a minimum first derivative with respect to time of the pulmonary artery pressure waveform.

8. The system of claim 7, wherein the second waveform feature is a pulmonary artery pulse pressure equal to a difference between the maximum pulmonary artery systolic pressure and the minimum pulmonary artery diastolic pressure, and wherein the hardware processor executes the right ventricular prediction software to determine the risk score based on the at least one first waveform feature and the pulmonary artery pulse pressure.

9. The system of claim 6, wherein the second waveform feature is a maximum pulmonary artery systolic pressure, and wherein the hardware processor executes the right ventricular prediction software to:
determine a pulse transit time equal to an elapsed time between a maximum right ventricular systolic pressure and the maximum pulmonary artery systolic pressure and
determine the risk score based on the at least one first waveform feature and the pulse transit time.

10. The system of claim 6, wherein the second waveform feature is a maximum pulmonary artery systolic pressure, and wherein the hardware processor executes the right ventricular prediction software to:
determine a systolic gradient equal to a pressure difference between a maximum right ventricular systolic pressure and the maximum pulmonary artery systolic pressure; and
determine the risk score based on the at least one waveform feature and the systolic gradient.

11. The system of claim 6, and further comprising a third hemodynamic sensor that produces, on an ongoing basis, a third hemodynamic sensor signal representative of one of a mixed venous oxygen saturation, a tissue oxygen saturation, and a cardiac output of the patient, wherein the hardware processor executes the right ventricular prediction software code to determine the risk score based on the at least one first waveform feature, the at least one second waveform feature, and the third hemodynamic sensor signal.

12. The system of claim 11, and further comprising a fourth hemodynamic sensor that produces, on an ongoing basis, a fourth hemodynamic sensor signal representative of a tissue oxygen saturation of the patient, wherein the third hemodynamic sensor signal is representative of the mixed venous oxygen saturation of the patient, and wherein the hardware processor executes the right ventricular prediction software code to determine the risk score based on the at least one first waveform feature, the at least one second waveform feature, the third hemodynamic sensor signal, and the fourth hemodynamic sensor signal.

13. The system of claim 12, and further comprising a fifth hemodynamic sensor that produces, on an ongoing, bases a fifth hemodynamic sensor signal representative of a cardiac output of the patient, wherein the hardware processor executes the right ventricular prediction software code to determine the risk score based on the at least one first waveform feature, the at least one second waveform feature, the third hemodynamic sensor signal, the fourth hemodynamic sensor signal, and the fifth hemodynamic sensor signal.

14. The system of claim 13, and further comprising a catheter inserted within a pulmonary artery and the right ventricle of the patient, wherein the first hemodynamic sensor, the second hemodynamic sensor, and the third hemodynamic senor are connected to the catheter, and wherein the fifth hemodynamic sensor includes a thermistor embedded within the catheter.

15. The system of claim 14, wherein the fourth hemodynamic sensor is a brain tissue oximetry sensor, and wherein the fourth sensor signal is representative of a cerebral tissue oxygen saturation of the patient.

16. The system of claim 13, wherein the hardware processor executes the right ventricular prediction software code to:
determine at least one of a continuous cardiac output, a stroke volume, a right ventricular ejection fraction, a right ventricular end diastolic volume, a pulse rate, and a blood temperature; and
determine the risk score based on the at least one first waveform feature, the at least one second waveform feature, the third hemodynamic sensor signal, the fourth hemodynamic sensor signal, the fifth hemodynamic sensor signal, and the at least one of a continuous cardiac output, a stroke volume, a right ventricular ejection fraction, a right ventricular end diastolic volume, a pulse rate, and a blood temperature.

17. The system of claim 13, wherein the hardware processor executes the right ventricular prediction software code to:
determine at least one of an arterial elastance, a maximal elastance, a ventriculoarterial coupling, a pulmonary vascular resistance, a pulmonary artery pulsatility index, a right ventricular function index; and
determine the risk score based on the at least one waveform feature, the at least one second waveform feature, the third hemodynamic sensor signal, the fourth hemodynamic sensor signal, the fifth hemodynamic sensor signal, and the at least one of at least one of the arterial elastance, the maximal elastance, the ventriculoarterial coupling, the pulmonary vascular resistance, the pulmonary artery pulsatility index, the right ventricular function index.

18. The system of claim 12, wherein the hardware processor executes the right ventricular prediction software code to determine the risk score based on:
at least one of a right ventricular end diastolic pressure, a right ventricular diastolic gradient, and a minimum first derivative with respect to time of the right ventricular pressure waveform;
at least one of a maximum first derivative with respect to time of the right ventricular pressure waveform, a right ventricular systolic pressure, a right ventricular end systolic pressure, and a right ventricular pulse pressure;
at least one of a right ventricular diastolic pressure and a right ventricular pulse pressure variation;
at least one of a pulse transit time, a mean pulmonary artery pressure, and a systolic gradient; and
at least one of a mixed venous oxygen saturation and a cerebral oxygen saturation.

19. The system of claim 1, wherein the hardware processor executes the right ventricular prediction software code to subdivide a range of risk scores into at least three continuous and sequential subranges, wherein a first subrange is predictive of right ventricular dysfunction of the patient, and wherein a second subrange of risk scores is indicative of potential right ventricular dysfunction, and wherein a third subrange of risk scores is indicative of a stable patient.

20. The system of claim 1, wherein the user interface includes a sensory alarm, and wherein the hardware processor executes the right ventricular prediction software code to activate the sensory alarm when the risk score is within the first subrange.
